(19) United States
(12) Patent Application Publication
(10) Pub. No.: US 2026/0060946 A1
(43) Pub. Date: Mar. 5, 2026

(54) NUCLEAR RECEPTOR SUBFAMILY 4 GROUP A AGONISTS AND METHODS OF USE FOR TREATMENT OF INFECTIOUS DISEASES

(71) Applicants: TEXAS BIOMEDICAL RESEARCH INSTITUTE, San Antonio, TX (US); SOUTHWEST RESEARCH INSTITUTE, San Antonio, TX (US)

(72) Inventors: Larry S. Schlesinger, San Antonio, TX (US); Eusondia Arnett, San Antonio, TX (US); Jonathan Bohmann, San Antonio, TX (US); Christopher Dorsey, San Antonio, TX (US)

(21) Appl. No.: 19/312,265

(22) Filed: Aug. 27, 2025

Related U.S. Application Data
(60) Provisional application No. 63/687,541, filed on Aug. 27, 2024.

(51) Int. Cl. A61K 31/17; A61K 31/165; A61K 31/166; A61K 31/167; A61K 31/192; A61K 31/194; A61K 31/498; A61K 45/06

(52) U.S. Cl. CPC A61K 31/17; A61K 31/165; A61K 31/166; A61K 31/167; A61K 31/192; A61K 31/194; A61K 31/498; A61K 45/06

(57) ABSTRACT

Provided here are nuclear receptor 4A agonists and methods of use for treatment of an infectious disease or an inflammation-related disease. These agonists include 3-(adamantan-1-yl)-1-(2-hydroxy-2-phenylpropyl)urea (available at Life Chemicals as F6279-0659), N-(3-acetylphenyl)-5,6,7,8-tetrahydronaphthalene-2-carboxamide (available at Life Chemicals as F0537-0485), N-[(1-hydroxy-2,3-dihydro-1H-inden-1-yl)methyl]naphthalene-1-carboxamide (available at Life Chemicals as F5857-5371), 3,4-difluoro-N-[(2-hydroxy-1,2,3,4-tetrahydronaphthalen-2-yl)methyl]benzamide (available at Life Chemicals as F6414-1064), N-(3-hydroxy-3-phenylpropyl)naphthalene-1-carboxamide (available at Life Chemicals as F5857-6887), N-(2-hydroxy-3-phenylpropyl)naphthalene-1-carboxamide (available at Life Chemicals as F6200-1091) or a pharmaceutically acceptable salt thereof. These agonists include one or more of F5964-0242, F5964-0253, F1065-0517, F0020-1803, F5857-5401, F0915-2916, F0307-0288, F3350-0754, F6172-0216, F3367-0092, F6172-0224, F5857-2441, F5857-6404, and F5857-5470 (available at Life Chemicals) or a pharmaceutically acceptable salt or derivative thereof.

### GOVERNMENT SUPPORT

This invention was made with government support under grant number: 1R01AI136831-01AI awarded by the National Institutes of Health. The government has certain rights in the invention.

### TECHNICAL FIELD

The disclosure relates to certain agonists of the nuclear receptor 4A family (NR4As) and methods of their use in the treatment of an infectious disease or an inflammation-related disease.

### BACKGROUND

Lungs are constantly exposed to pollutants, allergens, and microbes. Alveolar macrophages (AMs) must clear inhaled particulates without damaging the alveoli; thus, they possess a unique, highly regulated immune response that remains poorly understood, especially for human AMs (HAMs). Macrophage nuclear receptors (NRs) act as major ligand-activated transcriptional regulators of inflammation and are bona fide targets for many FDA-approved drugs. Anti-apoptotic MCL-1 was identified as an effector of the NR peroxisome proliferator-activated receptor gamma (PPARγ) that is critical for growth of Mycobacterium tuberculosis (M.tb). MCL-1 inhibitors that are in clinical trials and a FDA approved BCL-2 specific inhibitor robustly inhibit drug-sensitive and-resistant M.tb growth in human macrophages and a human in vitro granuloma model (US Patent Publication US20240180929); thus, demonstrating efficacy and viability of a potential targeted host-directed therapy (HDT) approach for tuberculosis (TB) based on NRs and/or their effectors.

Current TB treatments involve a cocktail of antibiotics for 4-6 months, although there have been recent efforts to shorten this to 1-6 months. Drug resistant TB treatment is even more challenging, requiring additional second line drugs for a much longer duration with more side effects and cost, and with low success rates (50-75%). New antibiotics for infectious agents face the problem of becoming obsolete due to rapid development of drug resistance, and the problem is compounded by the fact that pharmaceutical companies have largely withdrawn from antimicrobial development due to limited return on investment and other factors. Despite their promise, esp. for drug-resistant pathogens, development of HDTs for infectious diseases has lagged relative to other disease areas.

### SUMMARY

Provided here are compositions and methods to address these shortcomings of the art and provide other additional or alternative advantages. The disclosure herein provides one or more embodiments of NR4A agonists and methods for treatment of an infectious disease, such as tuberculosis, or an inflammation-related disease, such as diabetes, aging, Alzheimer's disease, or cancer. Certain embodiments include use of these agonists for the treatment of infectious diseases caused by M. tuberculosis. As NR4As are host molecules, their agonists are expected to limit growth of drug susceptible and resistant M. tuberculosis. Certain embodiments include use of these agonists for the treatment of infectious diseases caused by one or more intracellular pathogens, such as Salmonella, Leishmania, Listeria monocytogenes, Francisella tularensis, Yersinia pestis, Histoplasma, Cocciodomycosis, Blastomycocis, Coxiella burnetii, and viruses like COVID-19.

NR4A genes are highly expressed in alveolar macrophages, which are the primary immune cell in the lungs that inhaled pathogens like M. tuberculosis and SARS-CoV2 must evade. These pathogens are in the category of intracellular pathogens, which live in host cells such as macrophages. As evidenced by the data provided herein, activating NR4A1 or NR4A2 reduces M. tuberculosis burden in macrophages by 65% (FIG. 5), About 20 different NR4A agonists have been identified, many of which reduced M. tuberculosis growth in human macrophages, 4 by 75% or more (FIGS. 11, 22, and 26).

Embodiments of methods for treatment of an infectious disease or an inflammation-related disease include administering a therapeutically effective amount of a NR4A agonist. In some embodiments, these NR4A agonists contain a secondary alcohol group (—OH), connected to an alkyl chain (typically 2-3 carbons), which is attached to an amide nitrogen. In certain embodiments, these NR4A agonists have either Formula 1: R¹—CO—NH—CH₂—CHR²—OH or Formula 2: R¹—CO—NH—CHR²—CH₂OH, where R¹=a cyclic component (including but not limited to aromatic components, such as naphthalene, tetrahydronaphthalene, difluorobenzene, or based on cyclic alkanes, such as adamantyl-urea) and R²=hydrogen, phenyl, or part of a cyclic component. In certain embodiments, these NR4A agonists contain an amide bond or a urea linkage. In certain embodiments, these NR4A agonists contain at least one cyclic component such as aromatic ring component or based on a cyclic alkane. The aromatic ring component can be based on naphthalene or substituted naphthalene. In certain embodiments, these NR4A agonists contain an alkyl linker connecting the cyclic component to the amide/urea functionality.

Embodiments of methods for treatment of an infectious disease or an inflammation-related disease include administering a therapeutically effective amount of 3-(adamantan-1-yl)-1-(2-hydroxy-2-phenylpropyl)urea (available at Life Chemicals as F6279-0659) or a pharmaceutically acceptable salt thereof. Embodiments of methods for treatment of an infectious disease or an inflammation-related disease include administering a therapeutically effective amount of N-(3-acetylphenyl)-5,6,7,8-tetrahydronaphthalene-2-carboxamide (available at Life Chemicals as F0537-0485) or a pharmaceutically acceptable salt thereof. Embodiments of methods for treatment of an infectious disease or an inflammation-related disease include administering a therapeutically effective amount of N-[(1-hydroxy-2,3-dihydro-1H-inden-1-yl)methyl]naphthalene-1-carboxamide (available at Life Chemicals as F5857-5371) or a pharmaceutically acceptable salt thereof. Embodiments of methods for treatment of an infectious disease or an inflammation-related disease include administering a therapeutically effective amount of 3,4-difluoro-N-[(2-hydroxy-1,2,3,4-tetrahydronaphthalen-2-yl)methyl]benzamide (available at Life Chemicals as F6414-1064) or a pharmaceutically acceptable salt thereof. Embodiments of methods for treatment of an infectious disease or an inflammation-related disease include administering a therapeutically effective amount of N-(3-hydroxy-3-phenylpropyl)naphthalene-1-carboxamide (available at Life Chemicals as F5857-6887) or a pharmaceutically acceptable salt thereof. Embodiments of methods for treatment of an infectious disease or an inflammation-related disease include administering a therapeutically effective amount of N-(2-hydroxy-3-phenylpropyl)naphthalene-1-carboxamide (available at Life Chemicals as F6200-1091) or a pharmaceutically acceptable salt thereof. Embodiments of methods for treatment of an infectious disease or an inflammation-related disease include administering a therapeutically effective amount of one or more of F5964-0242, F5964-0253, F1065-0517, F0020-1803, F5857-5401, F0915-2916, F0307-0288, F3350-0754, F6172-0216, F3367-0092, F6172-0224, F5857-2441, F5857-6404, and F5857-5470 (available at Life Chemicals).

Aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. These drawings illustrate the principles of the disclosure and no limitation of the scope of the disclosure is thereby intended.

FIGS. 1A and 1B are diagrammatic representation of the structure of NR4A Family Members. FIG. 1A depicts NRs consist of a DNA binding domain (DBD), ligand binding domain (LBD) and activation function (AF-1) that interacts at the promoter with coactivators to induce gene transcription. FIG. 1B depicts a schematic representation of NR4A1, 2 and 3 with their functional domains and % homology between the NRs, indicating 60% homology in the LBD. Numbers indicate amino acid positions of human proteins. Images are from Leopold Wager et al. 2019 PLoS Pathog (FIG. 1A) and Boulet et al. 2022 Curr Res Immunol (FIG. 1B).

FIGS. 2A-2D are graphical representations of the PPARγ and NR gene expression in HAMs. NR gene expression significantly decreases following removal from the lung. HAMs were isolated from human donors & isolated RNA was analyzed by AmpliSeq transcriptome analysis (counts are RNA abundancy), mean±SEM, each point indicates one donor. n=8. *p<0.05, **p<0.01, ****p<0.0001.

FIG. 3 is a graphical representation showing that NR4A1-3 expression are up-regulated in response to M.tb. MDMs (monocyte-derived macrophages) were infected with M.tb for 24 h and qRT-PCR was performed. Results are mean±SEM, n=11, *p<0.05, **p<0.01.

FIG. 4 is a graphical representation showing NR4A1-3 gene expression is increased in AMLs compared to MDMs. Basal expression was measured by qRT-PCR. Results are mean±SEM, n=7-8. *p<0.05, **p<0.01.

FIG. 5 is a graphical representation showing NR4A1, 2, and 3 limit M.tb growth. MDMs and AMLs were infected with M.tb-lux, then treated with 50 μM 6MP (NR4A1 agonist), 100 μM C-DIM12 (NR4A2 agonist), or 20 μM PGA2 (NR4A1 and NR4A3 agonist). After 4 days, M.tb luciferase activity was determined. Results are relative luminescence units (RLUs), mean±SD, representative of n=2, ***p<0.001, ****p<0.0001 relative to untreated.

FIGS. 6A-6D are graphical representations showing NR4A1 is important for M.tb control in mice. In FIGS. 6A-6B, WT or NR4A1 knock-out (KO) mice were aerosol infected with M.tb Erdman and sacrificed after 15 days. In FIGS. 6C-6D, C3HeB/FeJ mice were aerosol infected with M.tb Erdman, rested for 3 weeks, then treated with NR4A1 agonists CsnB, 6-MP or the solvent control PBS for 4 weeks. CFUs were determined in the lungs (A,C) & spleens (B,D). There were 7-8 mice/group, each dot is one mouse, n=1. Mean±SD. *p<0.05, **p<0.01, ***p<0.001, relative to PBS/WT control.

FIGS. 7A-7B depict that NR4A1 agonist 6-MP controls M.tb in a Lung on a Chip (LoC) model (as described in Mishra et al. 2023 Cell). AMLs and alveolar epithelial cells seeded to the LoC were infected with fluorescent M.tb (red) and treated with NR4A1 agonist 6-MP. FIG. 7B, left panel depicts M.tb growth in AMLs and ATs as determined by assessing microcolony size. FIG. 7B, right panel depicts AMLs and ATs (treated with NR4A1 agonist 6-MP) were stained for nuclei (dapi/blue) and imaged.

FIGS. 8A and 8B are graphical representations showing that NR4A1, NR4A2, and NR4A3 knockdown (KD) increases M.tb growth in human (FIG. 8A) and murine (FIG, 8B) macrophages.

FIG. 9 is a representative example of a compound of interest docked with NR4A2. F0537-0485 (Life Chemicals, yellow stick) docked with the NR4A2 ligand binding domain (green), within the narrow binding pocket indicated with a yellow arrow.

FIG. 10 is a diagrammatic representation of the model used herein for developing, refining, and testing novel compounds. At step 1, putative compounds are assessed for NR4A transcriptional activity and those that drive NR4A activity are tested in step 2 in human and murine macrophages. In step 3 and step 4, the compounds were evaluated in a human granuloma model and LoC model, respectively, to assess ability to control M.tb growth. After iterative rounds of testing and refining, at step 5, the top compounds (that are specific for NR4As, reduce M.tb growth the most, cause no macrophage toxicity, have high water solubility and oral availability, etc.) are tested for activity in M.tb-infected mice. Only compounds that reduce M.tb growth>70% are selected.

FIGS. 11A and 11B are graphical representations showing that certain putative NR4A1 & 2 agonists reduce M.tb growth in human MDMs and murine bone marrow derived macrophages, respectively. Human MDMs (FIG. 11A) and murine bone marrow derived macrophages (FIG. 11B) were infected with M.tb-lux, then treated with the indicated compounds (50 μM) for 4 days. M.tb growth was assessed as RLUs. M.tb growth relative to the solvent control (−), mean±SEM, n=2, *p<0.05, **p<0.01, ***p<0.001 relative to (−). In FIG. 11A, the compounds are listed based on which NR4A they are predicted to bind (1, 1&2, or 2), compounds with amide or urea groups are in white, and those with carboxylic acids are in gray. Compounds are: A (F5964-0242), B (F6279-0659), C (F5964-0253), D (F1065-0517), E (F0020-1803), F (F5857-5371), G (F5857-5401), H (F0915-2916), I (F0537-0485), and J (F0307-0288).

FIGS. 12A-12E, FIGS. 13A-13E, and FIGS. 14A-14E are graphical representations of the three compounds that significantly limited M.tb growth without causing macrophage toxicity—two compounds by >80%: B (F6279-0659) (FIGS. 12A-12E) and I (F0537-0485) (FIGS. 13A-13E); and one by 50%: F (F5857-5371) (FIGS. 14A-14E), respectively. M.tb growth was assessed by M.tb luciferase activity (RLUs) daily. M.tb growth was assessed by colony forming unit (CFU) after 7 days (FIG. 12C). Macrophage toxicity was assessed by LDH release assay. The two experiments were done with macrophages isolated from two different human donors.f

FIGS. 15A-15E, FIGS. 16A-16E, FIGS. 17A-17E, FIGS. 18A-18D, FIGS. 19A-19D, FIGS. 20A-20E, and FIGS. 21A-21D are graphical representations of the seven compounds that had no significant impact on M.tb growth growth—A (F5964-0242), C (F5964-0253), D (F1065-0517), E (F0020-1803), G (F5857-5401), H (F0915-2916), and J (F0307-0288), respectively. M.tb growth was assessed by M.tb luciferase activity (RLUs) daily. M.tb growth was assessed by colony forming unit (CFU) after 7 days (FIG. 15C and FIG. 16C). Macrophage toxicity was assessed by LDH release assay. The two experiments were done with macrophages isolated from two different human donors.

FIGS. 22A-22B are graphical representations of the effect of the 10 compounds on M.tb growth in human macrophages at day 4 and day 7 at different concentrations (0 μM, 5 μM, 15 μM, and 50 μM).

FIGS. 23A-23D, FIGS. 24A-24D, and FIGS. 25A-25B are graphical representations of the second batch of 10 compounds for anti-M.tb activity (left panel) and macrophage toxicity (right panel) in human macrophages.

FIGS. 26A-26D are graphical representations of the effect of the 20 compounds on M.tb growth in human macrophages at day 4 (FIGS. 26A-26B) and day 7 (FIGS. 26C-26D) at 50 μM.

FIGS. 27A-27E are graphical representations of the effect of the B, F, and I compounds on anti-M.tb activity in murine macrophages, as assessed by luciferase activity, with 6MP (a NR4A agonist) as a control.

### DETAILED DESCRIPTION

The NR4A family is a subgroup of NRs linked to cancer, aging, diabetes and the immune response, however prior to the disclosure herein, it is unknown how the NR4As control infection, and if they can be targeted to treat infectious diseases, such as tuberculosis (TB) and COVID which together killed over 3.3 million people in 2020.

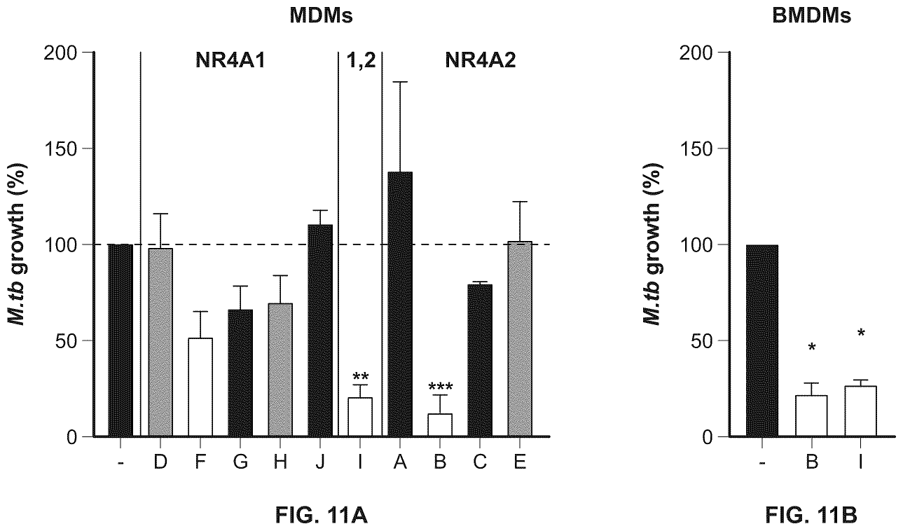

The disclosure herein provides one or more embodiments of NR4A agonists and methods for treatment of an infectious disease, such as tuberculosis, or an inflammation-related disease, such as diabetes, aging, Alzheimer's disease, or cancer. Certain embodiments include use of these agonists for the treatment of infectious diseases caused by M. tuberculosis. As NR4As are host molecules, their agonists are expected to limit growth of drug susceptible and resistant M. tuberculosis. Certain embodiments include use of these agonists for the treatment of infectious diseases caused by one or more intracellular pathogens, such as Salmonella, Leishmania, Listeria monocytogenes, Francisella tularensis, Yersinia pestis, Histoplasma, Cocciodomycosis, Blastomycocis, Coxiella burnetii, and viruses like COVID-19. About 20 different NR4A agonists have been identified, many of which reduced M. tuberculosis growth in human macrophages, 4 by 75% or more (FIGS. 11, 22, and 26).

Embodiments of methods for treatment of an infectious disease or an inflammation-related disease include administering a therapeutically effective amount of a NR4A agonist. In some embodiments, these NR4A agonists contain a secondary alcohol group (—OH), connected to an alkyl chain (typically 2-3 carbons), which is attached to an amide nitrogen. In certain embodiments, these NR4A agonists have either Formula 1: R¹—CO—NH—CH₂—CHR²—OH or Formula 2: R¹—CO—NH—CHR²—CH₂OH, where R¹=a cyclic component (including but not limited to aromatic components, such as naphthalene, tetrahydronaphthalene, difluorobenzene, or based on cyclic alkanes, such as adamantyl-urea) and R²=hydrogen, phenyl, or part of a cyclic component. In certain embodiments, these NR4A agonists contain an amide bond or a urea linkage. In certain embodiments, these NR4A agonists contain at least one cyclic component such as aromatic ring component or based on a cyclic alkane. The aromatic ring component can be based on naphthalene or substituted naphthalene. In certain embodiments, these NR4A agonists contain an alkyl linker connecting the cyclic component to the amide/urea functionality.

Embodiments of methods for treatment of an infectious disease or an inflammation-related disease include administering a therapeutically effective amount of 3-(adamantan-1-yl)-1-(2-hydroxy-2-phenylpropyl)urea (available at Life Chemicals as F6279-0659) or a pharmaceutically acceptable salt thereof. Embodiments of methods for treatment of an infectious disease or an inflammation-related disease include administering a therapeutically effective amount of N-(3-acetylphenyl)-5,6,7,8-tetrahydronaphthalene-2-carboxamide (available at Life Chemicals as F0537-0485) or a pharmaceutically acceptable salt thereof. Embodiments of methods for treatment of an infectious disease or an inflammation-related disease include administering a therapeutically effective amount of N-[(1-hydroxy-2,3-dihydro-1H-inden-1-yl)methyl]naphthalene-1-carboxamide (available at Life Chemicals as F5857-5371) or a pharmaceutically acceptable salt thereof. Embodiments of methods for treatment of an infectious disease or an inflammation-related disease include administering a therapeutically effective amount of 3,4-difluoro-N-[(2-hydroxy-1,2,3,4-tetrahydronaphthalen-2-yl)methyl]benzamide (available at Life Chemicals as F6414-1064) or a pharmaceutically acceptable salt thereof. Embodiments of methods for treatment of an infectious disease or an inflammation-related disease include administering a therapeutically effective amount of N-(3-hydroxy-3-phenylpropyl)naphthalene-1-carboxamide (available at Life Chemicals as F5857-6887) or a pharmaceutically acceptable salt thereof. Embodiments of methods for treatment of an infectious disease or an inflammation-related disease include administering a therapeutically effective amount of N-(2-hydroxy-3-phenylpropyl)naphthalene-1-carboxamide (available at Life Chemicals as F6200-1091) or a pharmaceutically acceptable salt thereof. Embodiments of methods for treatment of an infectious disease or an inflammation-related disease include administering a therapeutically effective amount of one or more of F5964-0242, F5964-0253, F1065-0517, F0020-1803, F5857-5401, F0915-2916, F0307-0288, F3367-0092, F3350-0754, F6172-0224, F6172-0216, F5857-2441, F5857-6404, F5857-5470 (available at Life Chemicals), and/or the derivatives of any of the foregoing.

The term “therapeutically effective amount” or “effective amount,” as used herein, refers to that amount of a composition, compound, or agent that imparts a modulating effect, which, for example, can be a beneficial effect, to a subject afflicted with an infectious disease, including improvement in the condition of the subject (e.g., in one or more symptoms), delay or reduction in the progression of the condition, prevention or delay of the onset of the disease, and/or change in clinical parameters, disease or illness, etc. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat” or “treating” or “treatment” refers to any type of action that imparts a modulating effect, which, for example, can be a beneficial effect, to a subject afflicted with a disease or illness, including improvement in the condition of the subject (e.g., in one or more symptoms), delay or reduction in the progression of the condition, and/or change in clinical parameters, disease or illness, etc.

“Pharmaceutically acceptable,” as used herein, means a material that is not biologically or otherwise undesirable, i.e., the material can be administered to an individual along with the compositions or agonists disclosed herein, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21st ed. 2005).

Embodiments also include formulations of pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

An emerging NR family (NR4A1, 2, and 3) was identified. Members of this family have high expression in AMs, similar to PPARγ (required for AM development), which swiftly declines after AM removal from the lung, indicating a role for the NR4A family in AM phenotype and function. Thus, the NR4A family and their downstream effectors are a new frontier for HDT approaches for TB. NR4As are important for control of M.tb in macrophages, epithelial cells, a lung on chip (LoC) model, and in vivo in mice. Thus, activation of NR4As and/or their effectors are targets for HDTs for TB.

M.tb upregulates NR4A and PPARγ activities in human macrophages with disparate effects on M.tb growth, suggesting an important balance between host defense (NR4As) and bacterial immune evasion (PPARγ). NR4A1 plays a protective role against M.tb. An in silico modelling technique and structure activity relationship (SAR)-guided studies were employed to identify novel specific NR4A agonists. Such modelling identified 6 compounds which bind NR4A1/2 and limit M.tb growth in macrophages.

The AM tissue environment is unique. AMs clear particulates/invaders while tightly regulating pro-inflammatory responses to maintain alveolar structure and enable gas-exchange. Considering this, the alveoli are relatively sluggish compared to other mucosal surfaces in controlling growth of air-borne infectious agents, particularly host-adapted intracellular pathogens of macrophages, such as M.tb that exploits the very nature of these cells.

NRs are critical sensors and regulators of immune functions and a bona fide class of proteins for FDA-approved drugs (not yet tested for TB). NRs are becoming more appreciated in M.tb pathogenesis but much is still unknown. The NR PPARγ is highly expressed in AMs and AMLs, and critical for AM development and function. PPARγ promotes M.tb growth in human macrophages and novel PPARγ effectors were identified with functional significance, including MCL-1, an anti-apoptotic BCL-2 family member (Arnet et al. 2018 PLoS Pathog). Targeting MCL-1 and BCL-2 robustly inhibits drug-sensitive and -resistant M.tb growth in monocyte-derived macrophages (MDMs) and a human in vitro granuloma model, providing the first evidence that targeting MCL-1/BCL-2 is a potential host directed therapy (HDT) for TB (Arnett et al. 2023 Biomed Pharmacother).

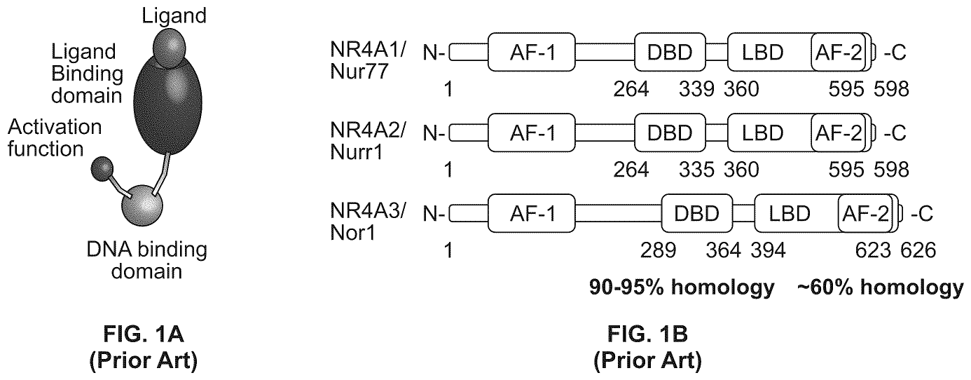

The NR4A family, comprised of NR4A1 (Nur77/TR3), NR4A2 (Nurr1) and NR4A3 (NOR1), shares structural homologies (FIG. 1; esp. NR4A1 and 2) and is highly conserved in the DNA-binding domain, but less so in the ligand-binding domain. They are linked to immunity, metabolism, apoptosis, and lipid uptake regulation. NRs operate in a tissue, gene, and signal-specific manner. FIGS. 1A and 1B are diagrammatic representation of the structure of NR4A Family Members. FIG. 1A depicts NRs consist of a DNA binding domain (DBD), ligand binding domain (LBD) and activation function (AF-1) that interacts at the promoter with coactivators to induce gene transcription. FIG. 1B depicts a schematic representation of NR4A1, 2 and 3 with their functional domains and % homology between the NRs. Numbers indicate amino acid positions of human proteins. Images are from Leopold Wager et al. 2019 PLoS Pathog (FIG. 1A) and Boulet et al. 2022 Curr Res Immunol (FIG. 1B).

The combination of host alveolar environment and M.tb factors make AMs permissive to M.tb. Use of the new AM-Like (AML) model that shares many AM characteristics (Pahari et al. 2024 STAR Protoc, Pahari et al. 2023 mBio) was used to interrogate NR4As' role(s) in HAMs. The LoC model was employed to study AML/AT interactions and bacterial growth at the liquid-air interface. NR4A agonists and WT and KO mouse models were used to investigate how NR4As shape the immune response to M.tb in vivo. NR4A1, 2, and 3 were evaluated to understand any redundant and/or antagonist effects of each NR4A family member in regulating macrophage and AT responses to M.tb. Modulation of macrophage NRs and their effectors is a promising approach for M.tb HDTs for both drug-susceptible and-resistant TB. Use of primary human macrophages (HAMs, AMLs, and monocyte-derived macrophages, MDMs) and epithelial cells for in vitro M.tb studies is critical for moving the field forward for this human-adapted pathogen and represents a proven strength of the labs. AMLs, MDMs and LoCs are tractable systems that allow for mechanistic studies of interactions between M.tb and the human host, critical for new knowledge of key elements of M.tb pathogenesis and the human response.

The first in vitro model for HAMs was developed and described in Pahari et al. 2024 STAR Protoc, and Pahari et al. 2023 mBio. AMLs demonstrated a similar phenotype to HAMs compared to MDMs including higher PPARγ and NR4A1-3 expression (FIG. 4, Pahari et al. 2023 mBio).

The NR4As bind to DNA as either monomers or homodimers. The NR4A ligand-binding pocket is small and filled with bulky side chains, suggesting that NR4A activity is regulated transcriptionally and post-translationally. The NR4As are considered immediate-early response genes that are induced in response to several factors (including M.tb) and act as global transcription regulators. They have been primarily studied in lymphocytes or cancer cell lines, and some work in macrophages indicates they have wide-ranging effects, including regulating expression of inflammatory genes. Despite being strongly conserved across species, NR4A1-3 have not been extensively studied in an infectious disease or an inflammation-related disease and have not been studied at all in the context of HAMs or ATs.

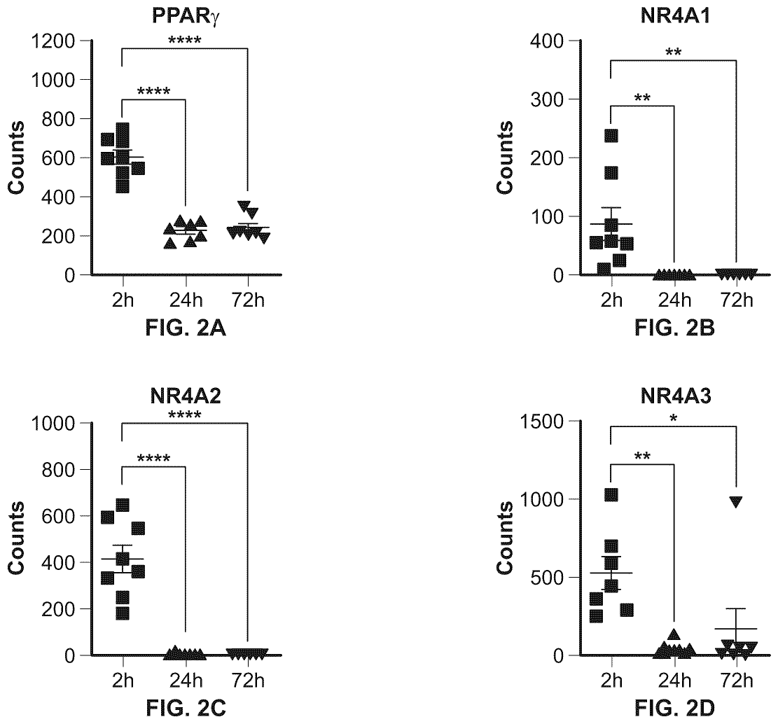

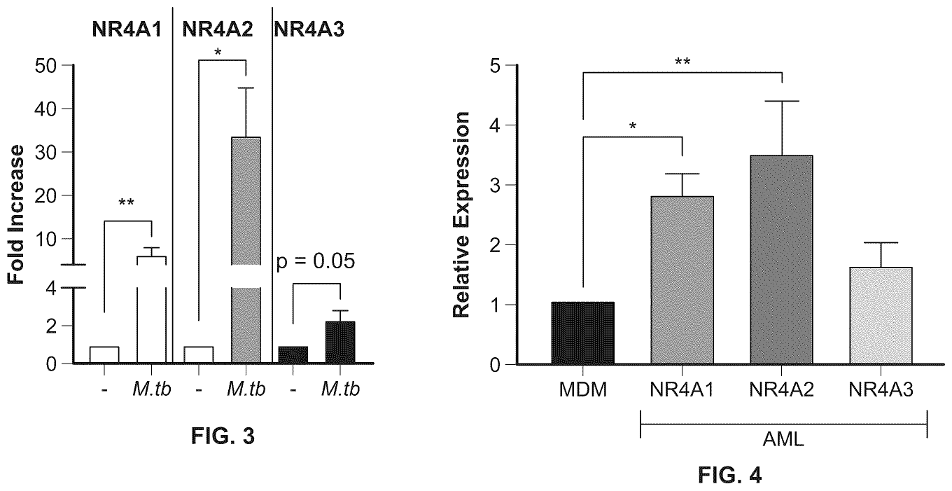

PPARγ and the NR4A family are highly expressed in freshly obtained HAMs and decrease markedly with time in culture. FIGS. 2A-2D are graphical representations of PPARγ and NR gene expression in HAMs. NR gene expression significantly decreases following removal from the lung. HAMs were isolated from human donors & isolated RNA was analyzed by AmpliSeq transcriptome analysis (counts are RNA abundancy), mean±SEM, each point indicates one donor. n=8. *p<0.05, **p<0.01, ****p<0.0001. NR4As, similar to PPARγ, may be important for HAM homeostasis and function. NR4A1-3 are more highly expressed in HAMs and AMLs compared to MDMs (FIG. 4), similar to PPARγ. FIG. 3 is a graphical representation showing that NR4A1-3 expressions are up-regulated in response to M.tb. MDMs were infected with M.tb for 24 h and qRT-PCR was performed. Results are mean±SEM, n=11, *p<0.05, **p<0.01.

NR4A1-3 are upregulated in human macrophages in response to M.tb (FIG. 3). FIG. 4 is a graphical representation showing NR4A1-3 gene expression is increased in AMLs compared to MDMs. Basal expression was measured by qRT-PCR. Results are mean±SEM, n=7-8. *p<0.05, **p<0.01.

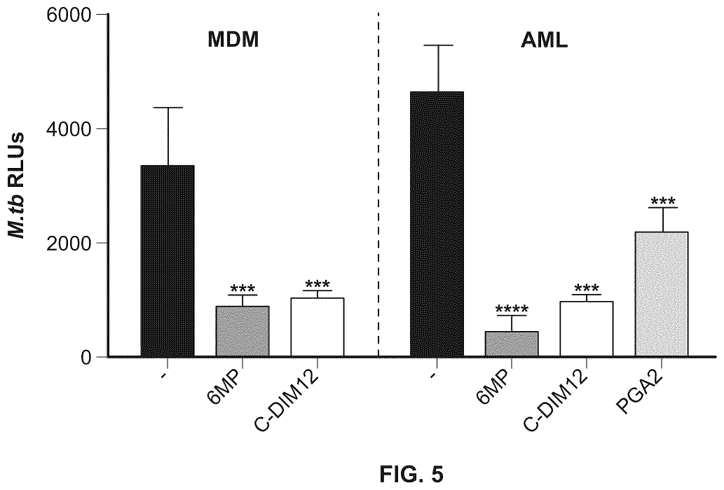

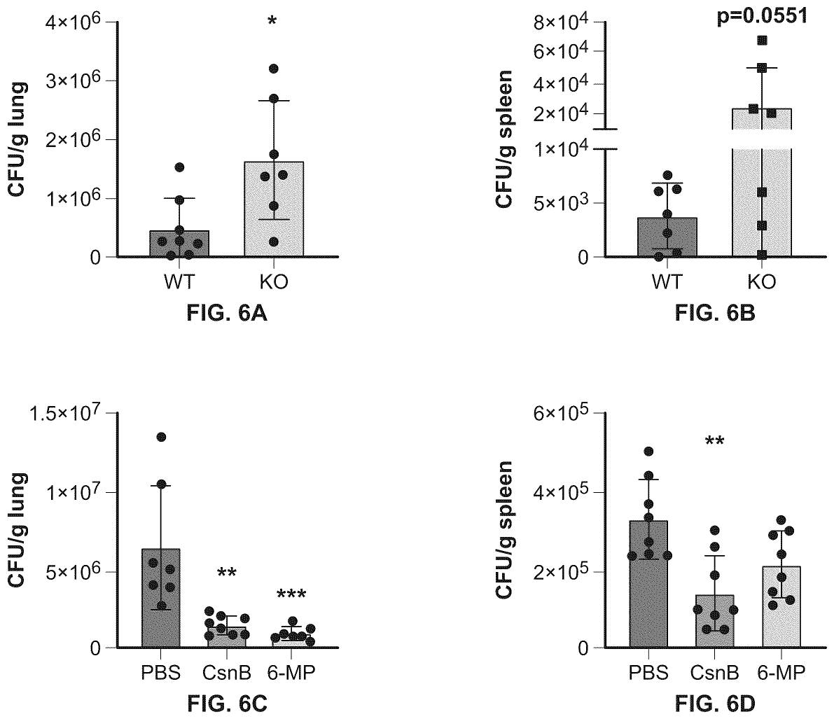

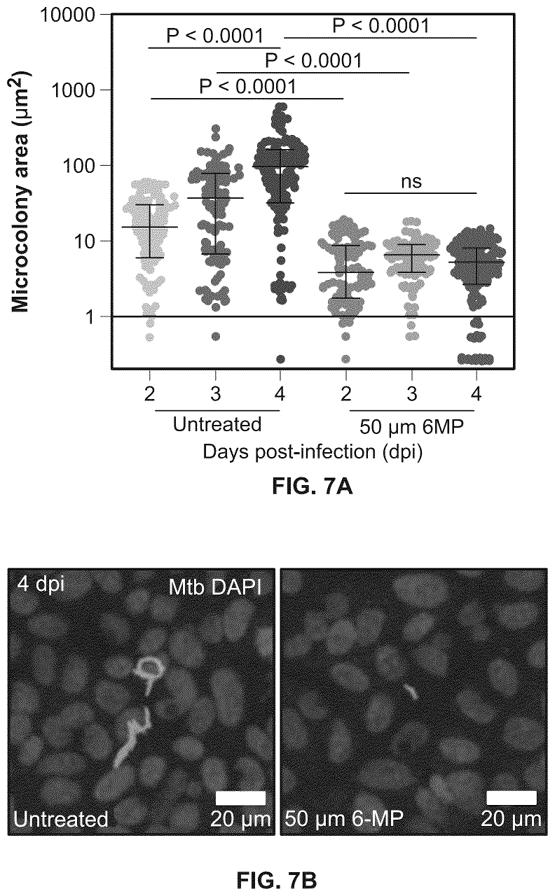

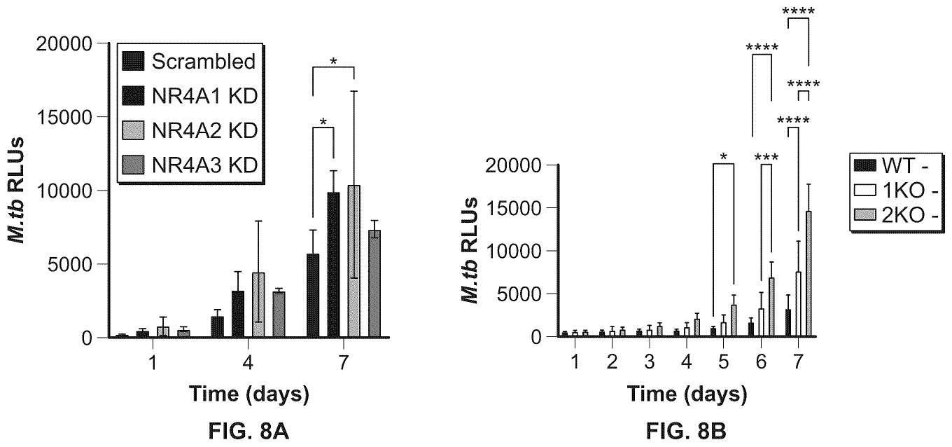

NR4A agonists reduce M.tb growth in macrophages, alone or cultured with ATs in a LoC, and in mice (FIGS. 5, 6A-6D, and 7A-7B) with similar results observed in murine bone marrow derived macrophages (BMDMs; FIG. 11B). Conversely, NR4A KD or KO increased M.tb growth in macrophages (FIGS. 8A-8B) and mice (FIGS. 6A-6D). FIG. 5 is a graphical representation showing NR4A1, 2, and 3 limit M.tb growth. Two different human macrophage models (MDMs and AMLs) were infected with M.tb-lux, then treated with 50 μM 6MP (NR4A1 agonist), 100 μM C-DIM12 (NR4A2 agonist), or 20 μM PGA2 (NR4A1 and NR4A3 agonist). After 4 days, M.tb luciferase activity was determined. Results are relative luminescence units (RLUs), mean±SD, representative of n=2, ***p<0.001, *p<0.0001 relative to untreated. FIGS. 6A-6D are graphical representations showing NR4A1 is important for M.tb control in mice. In FIGS. 6A-6B, WT or NR4A1 KO mice were aerosol infected with M.tb Erdman and sacrificed after 15 days. In FIGS. 6C-6D, C3HeB/FeJ mice were aerosol infected with M.tb Erdman, rested for 3 weeks, then treated with NR4A1 agonists CsnB, 6-MP, or the solvent control PBS for 4 weeks. CFUs were determined in the lungs (FIGS. 6A and 6C) & spleens (FIGS. 6B and 6D). 7-8 mice/group, each dot is one mouse, n=1. Mean±SD. *p<0.05, **p<0.01, **p<0.001, relative to PBS/WT control. FIGS. 7A-7B depict that NR4A1 agonist 6-MP controls M.tb in the LoC model. AMLs and ATs seeded to the LoC were infected with fluorescent M.tb (red) and treated with NR4A1 agonist 6-MP. FIG. 7B, left panel depicts M.tb growth in AMLs and ATs was determined by assessing microcolony size. FIG. 7B, right panel depicts AMLs and ATs were stained for nuclei (dapi/blue) and imaged. These findings indicate a host protective role for NR4A1-3 in restricting M.tb growth, antagonistic to PPARγ's known permissive role.

The human LoC model allows us to dissect differences between AT and AMLs in co-culture at an air-liquid interface and possible synergies or antagonisms in their responses brought about by AT/AM interactions. AMLs have high baseline PPARγ and NR4A1-3 expression, whereas PPARγ expression is low in both type I and type II ATs. Preliminary data from live-imaging studies in the LoC model show that continuous vascular perfusion of the NR4A1 agonist 6-MP decreased M.tb growth in both AMLs and ATs (FIGS. 7A-7B).

Preliminary in vitro data demonstrates the protective role of NR4As in human macrophage and epithelial responses to M.tb. NR4A1 is host protective: mice deficient in NR4A1 had higher bacterial growth in the lungs and spleen (FIGS. 6A-B) whereas mice treated with NR4A1 agonists had decreased bacterial burden (FIGS. 6C-6D).

NRs like PPARγ and the NR4As are promising targets for HDT (FIGS. 5, 6A-6D, and 7A-7B), However, the NR4As have small ligand binding domains (LBDs) and there are concerns about toxicity, solubility, specificity, and oral bioavailability of some currently available NR4A synthetic ligands (including CsnB and 6MP).

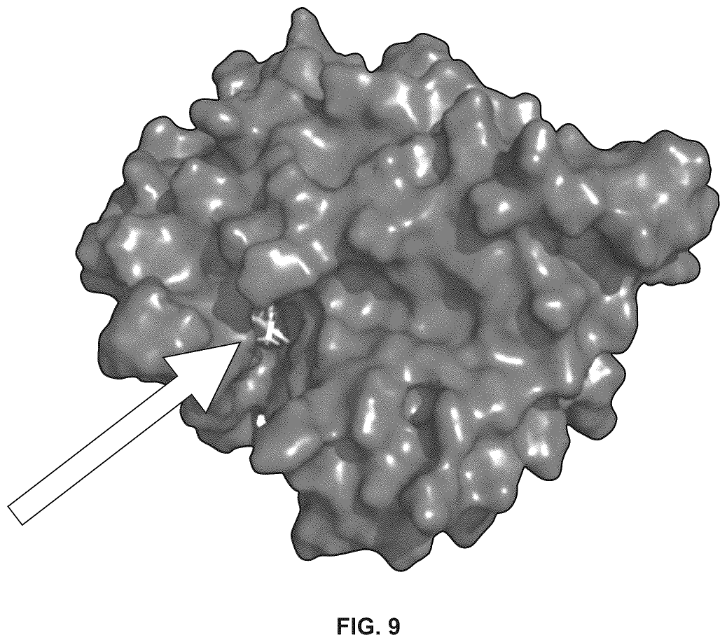

Virtual screens using Rhodium™ were conducted to model compound binding with the published NR4A1 and 2 LBD structures—the NR4As share 90% sequence homology in their DNA-binding domain, but only 60% in the LBD. Various crystal structures of both LBDs were obtained from the protein binding database and prepared for docking by removing co-crystallized ligands and peripheral water molecules. Commercially available compound libraries (Life Chemicals) were obtained and up to thirty 3D conformers of each compound were generated, including stereoisomers. These prepared libraries were then docked with each NR LBD crystal structure. Compounds were ranked and scored based on their predicted binding efficacy for each NR and predicted pharmacokinetic properties including cLogp, cPKa, and toxicological red flags, such as hERG and CYP inhibition using Stardrop's automated Derek Nexus algorithm. Initial analysis identified 10 compounds based on suggested binding scores that are predicted to dock in a well-defined pocket within the structure of either NR4A1 (compounds D, F, G, H, and J), NR4A2 (compounds A, B, C, and E) or both (compound I) LBDs (example in FIG. 9). For ease, compounds were lettered A-J, Life Chemicals product number is: A (F5964-0242), B (F6279-0659), C (F5964-0253), D (F1065-0517), E (F0020-1803), F (F5857-5371), G (F5857-5401), H (F0915-2916), I (F0537-0485) and J (F0307-0288).

Identity of ten compounds (Life Chemicals) were confirmed through liquid chromatography-mass spectrometry and nuclear magnetic resonance. For ease, compounds were lettered A-J, Life Chemicals product number is provided in parentheses: A (F5964-0242), B (F6279-0659), C (F5964-0253), D (F1065-0517), E (F0020-1803), F (F5857-5371), G (F5857-5401), H (F0915-2916), I (F0537-0485) and J (F0307-0288). Binding of 3 of the compounds with NR4A1/2 was confirmed using tryptophan quenching assays (as described in Karki et al. 2021 Cancers). Compound B bound NR4A2 with Kd=2.53 μM as compared to 9899 μM for NR4A1; Compounds F and I bound NR4A1 with Kd<5μM. Compounds were tested using Rhodium for putative binding to NR4A1 and NR4A2. These tests identified compounds with putative binding to NR4A1 and/or NR4A2. Compound I is shown in FIG. 9 being docked with the NR4A2 ligand binding domain, within the narrow binding pocket indicated by the yellow arrow.

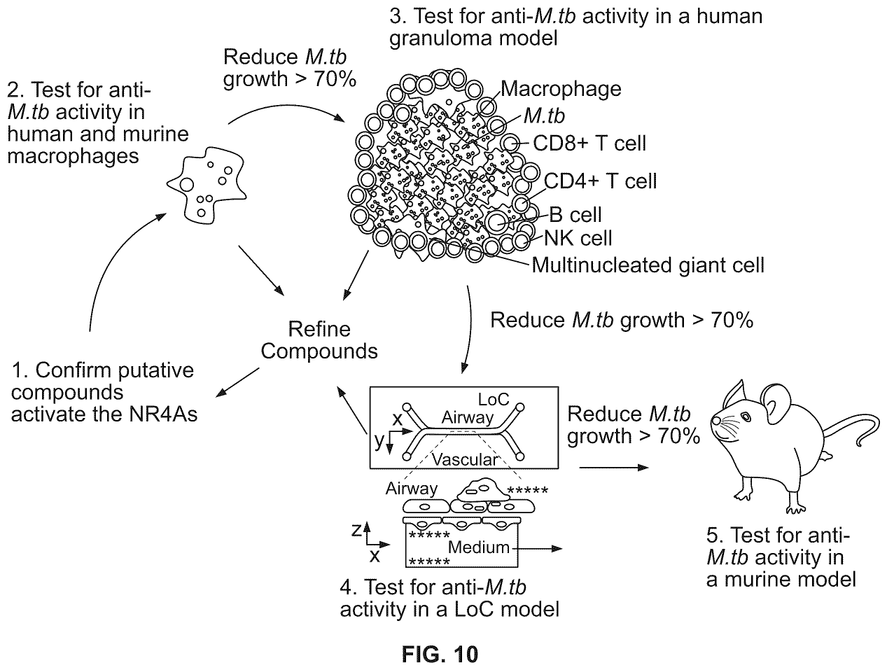

These compounds were tested for in vitro efficacy against M.tb in macrophages. As shown in FIG. 10, the assay for identifying TB therapeutics consists of identifying compounds active in human and murine macrophages (to determine if mice are a viable animal model for these compounds), then testing those compounds in the more complex human granuloma and LoC models for further down-selection. In the LoC model, the ability to cross the blood-air barrier is evaluated. Macrophages/LoCs/granulomas are infected with fluorescent or luminescent M.tb, then treated with the identified compounds and cell toxicity (LDH release assay) and M.tb growth (macrophages and granulomas: luciferase activity which correlates with M.tb growth; LoCs: microcolony area of fluorescent M.tb) are assessed over time. Activity of the compounds are evaluated in AMLs with higher NR4A expression (FIG. 4), akin to HAMs. Lead compounds are confirmed by CFU.

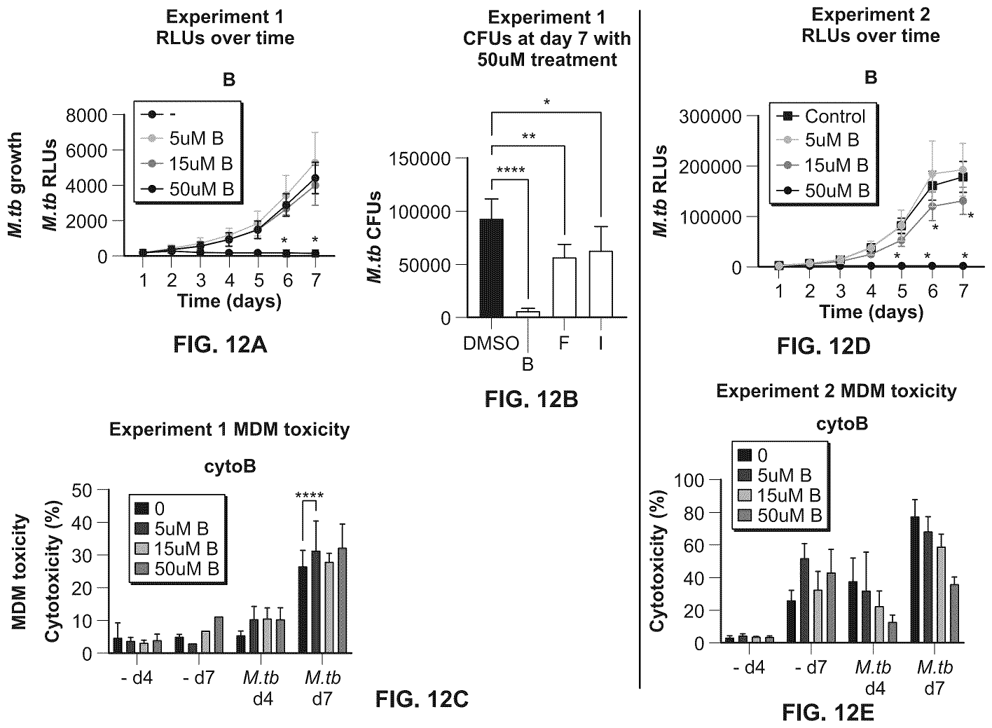

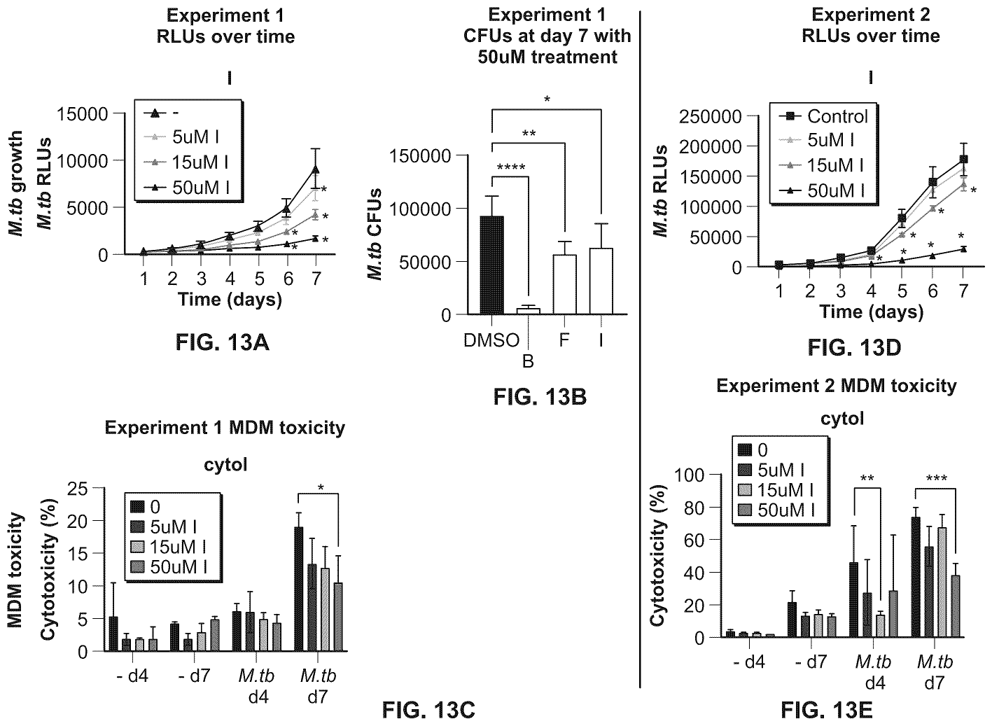

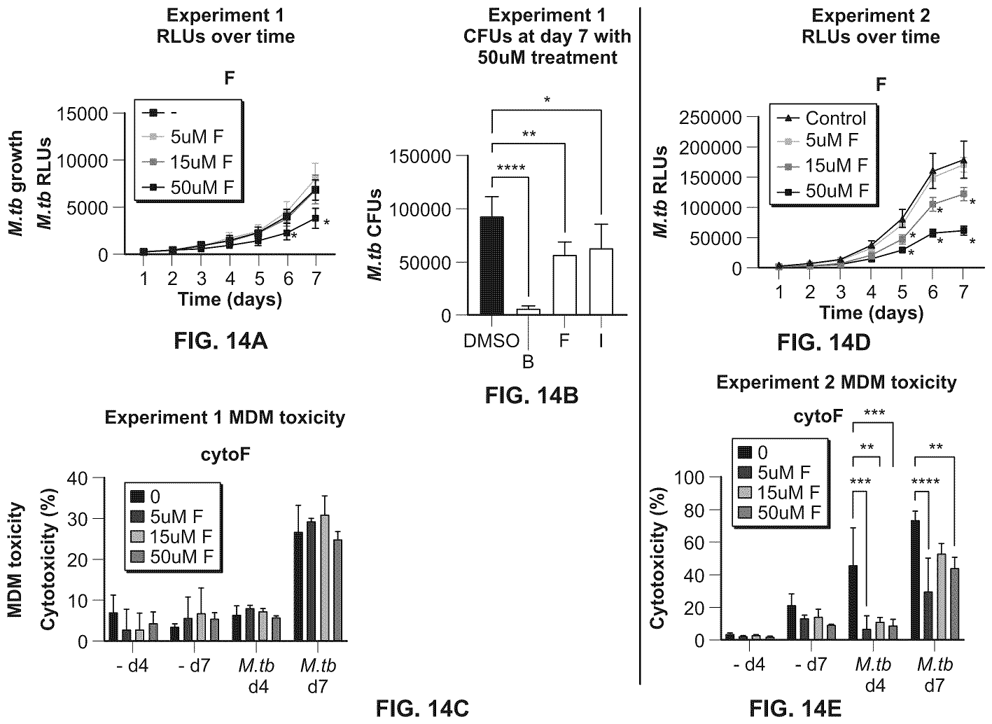

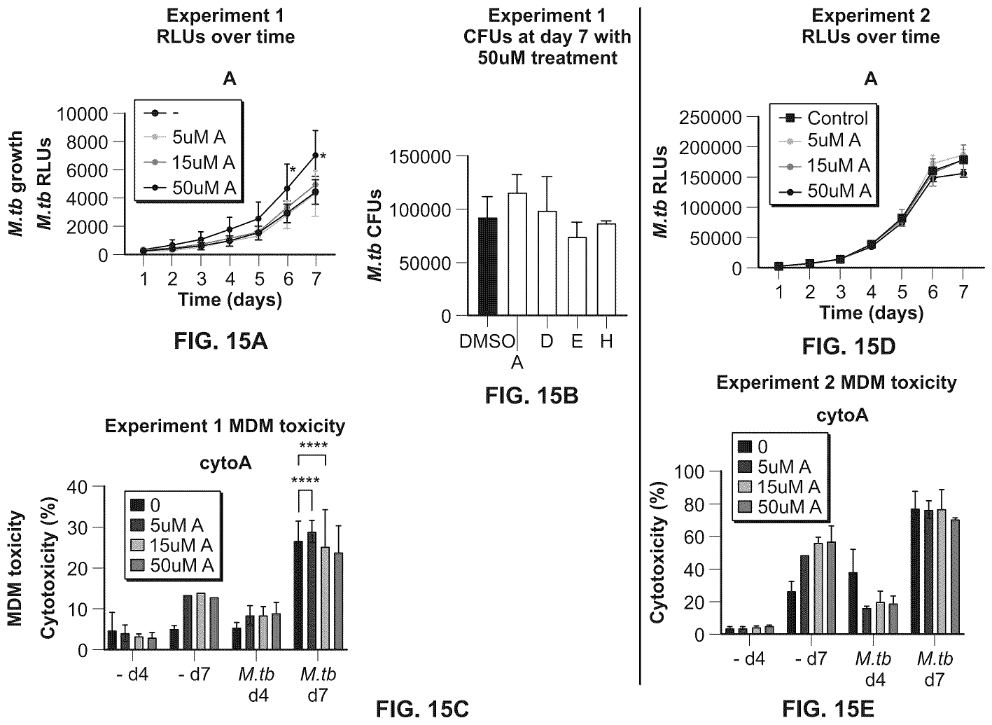

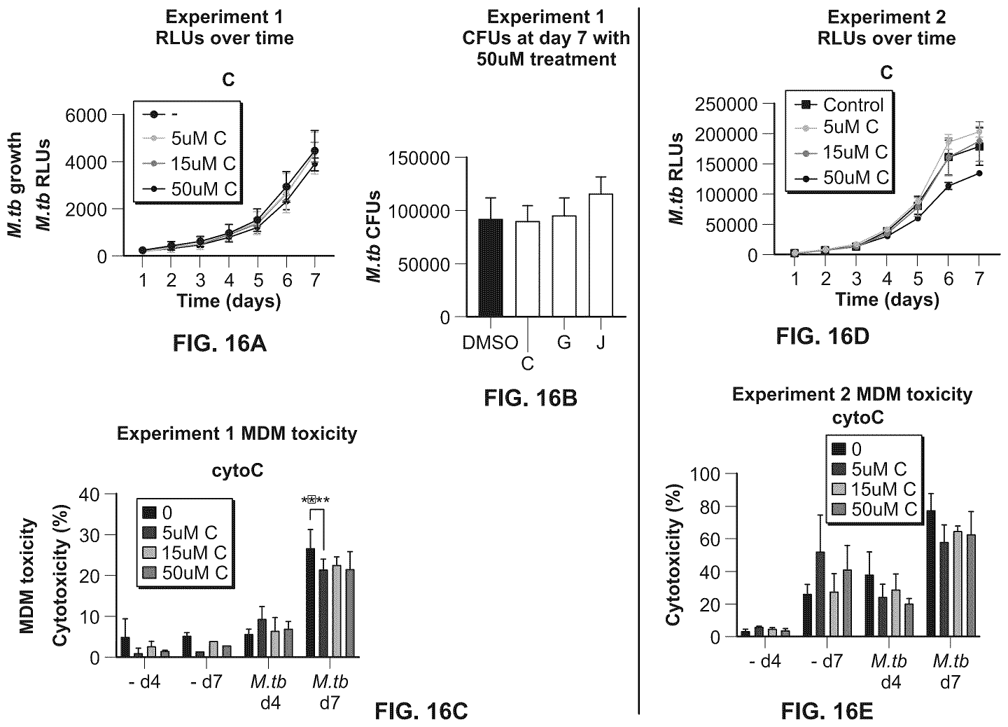

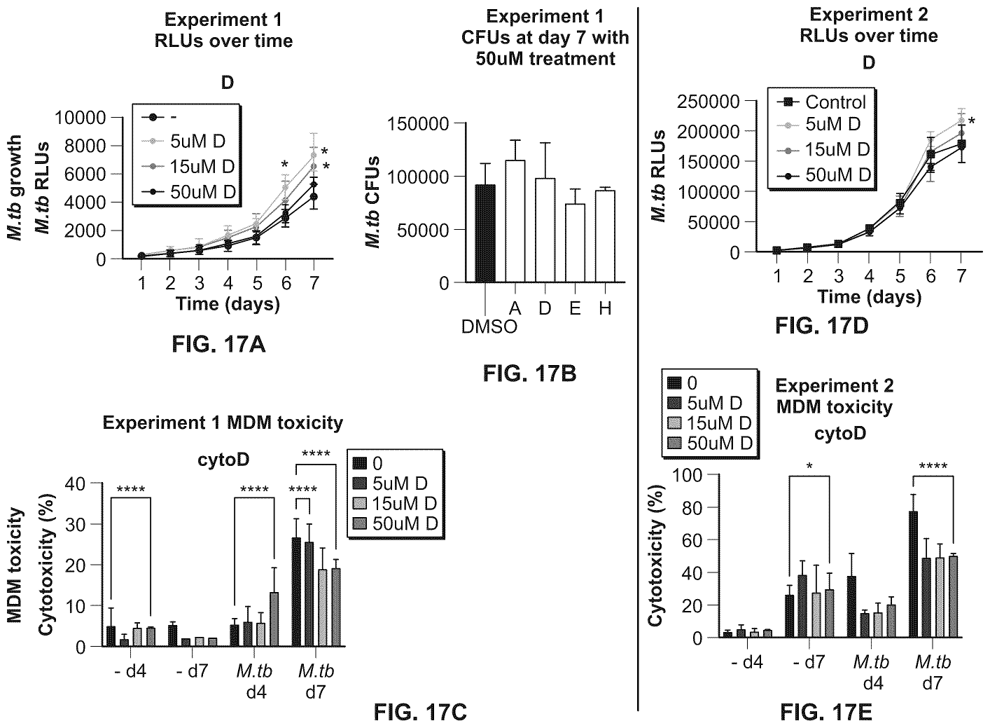

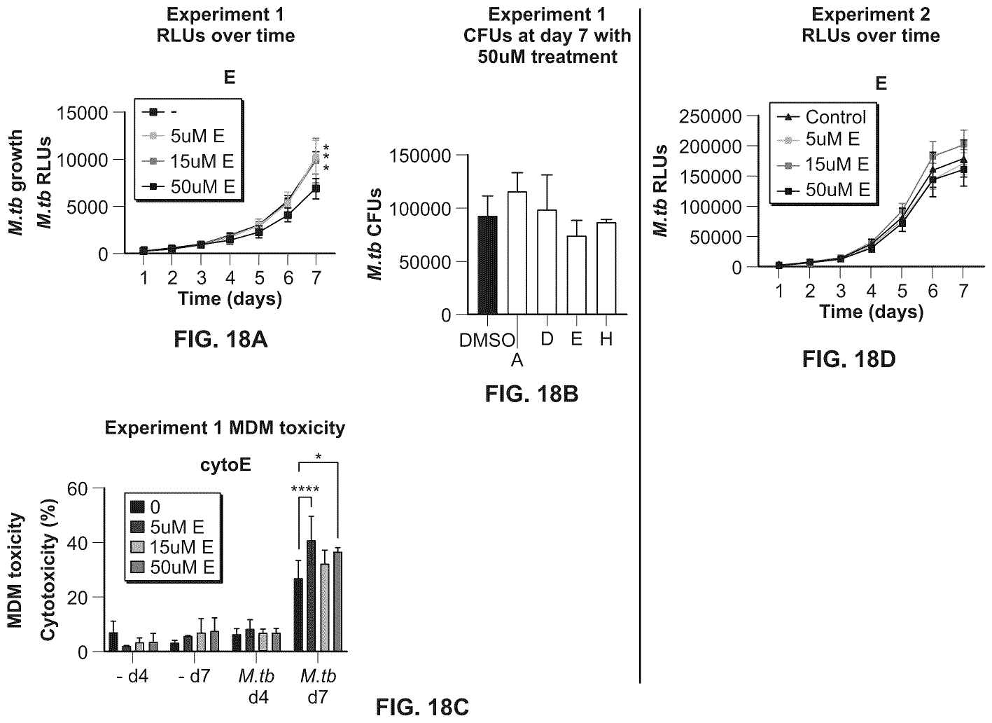

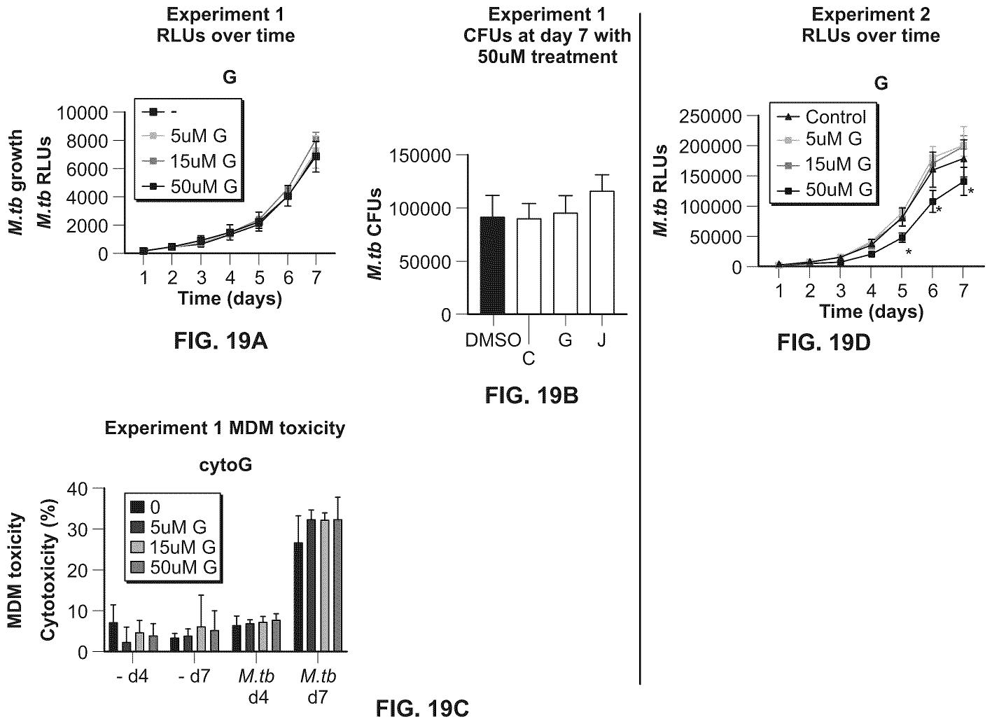

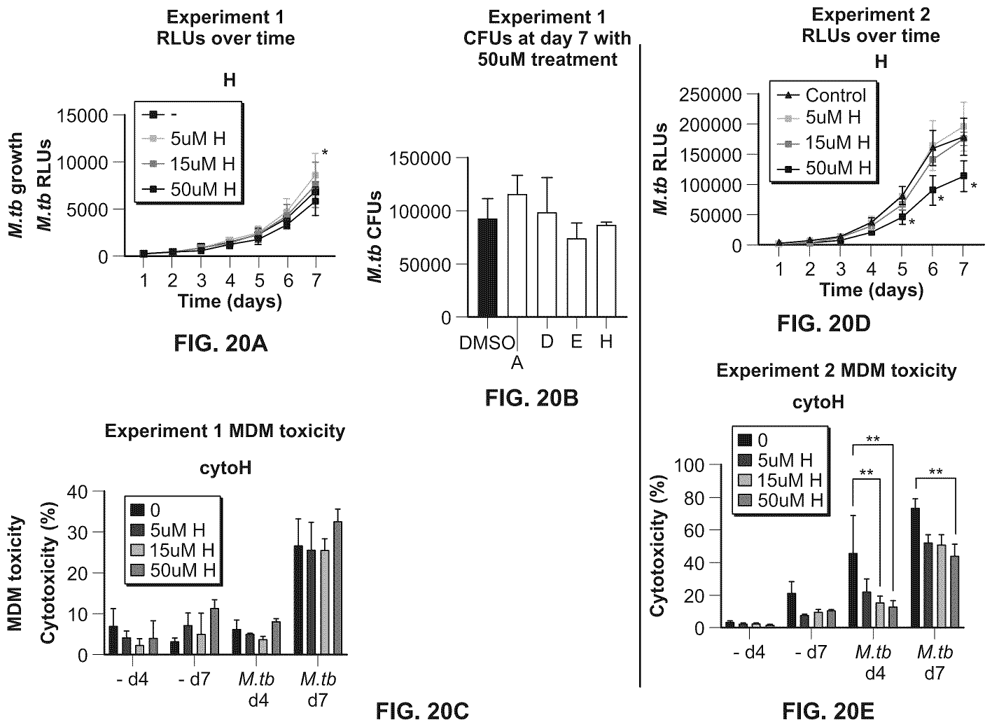

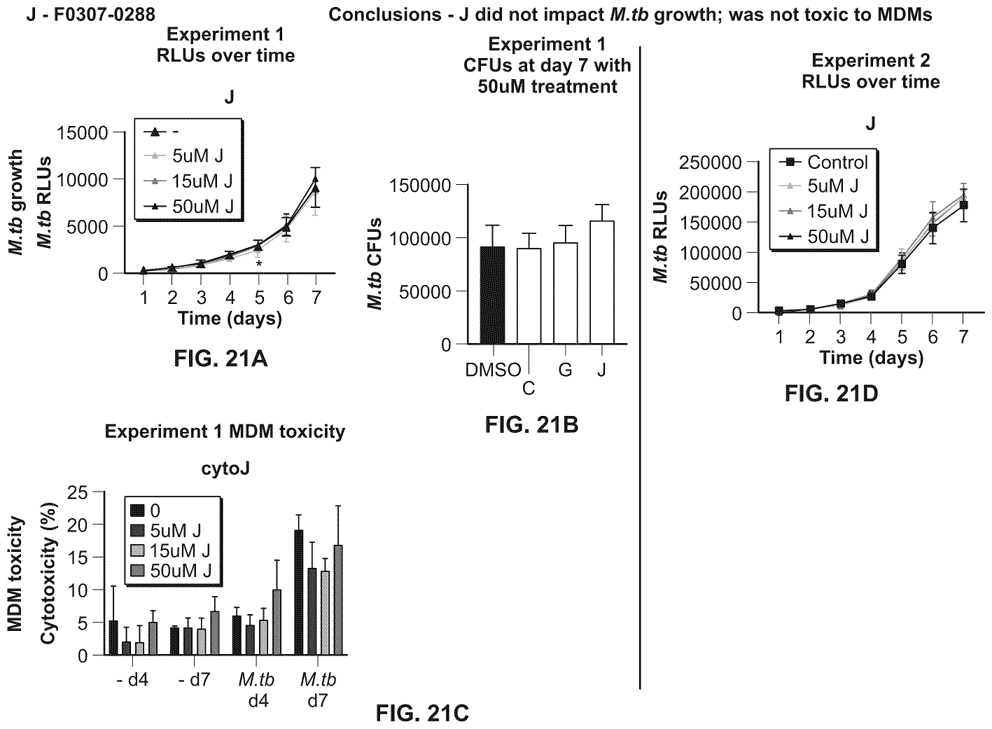

The ten compounds were evaluated for anti-M. tb activity and macrophage toxicity in human macrophages. M.tb growth was assessed by CFUs at day 7 and M.tb luciferase activity daily. MDM toxicity was assessed by LDH release assay. Three of the ten compounds significantly limited M.tb growth without causing MDM toxicity—two compounds by >80%: B (F6279-0659) and I (F0537-0485); and one by 50%: F (F5857-5371)—as shown in FIGS. 12-14, respectively. Seven of the ten compounds had no significant impact on M.tb growth—A (F5964-0242), C (F5964-0253), D (F1065-0517), E (F0020-1803), G (F5857-5401), H (F0915-2916), and J (F0307-0288)—as shown in FIGS. 15A-15E, FIGS. 16A-16E, FIGS. 17A-17E, FIGS. 18A-18D, FIGS. 19A-19D, FIGS. 20A-20E, and FIGS. 21A-21D, respectively. Only one compound was toxic to macrophages, and some reduced M.tb-driven toxicity.

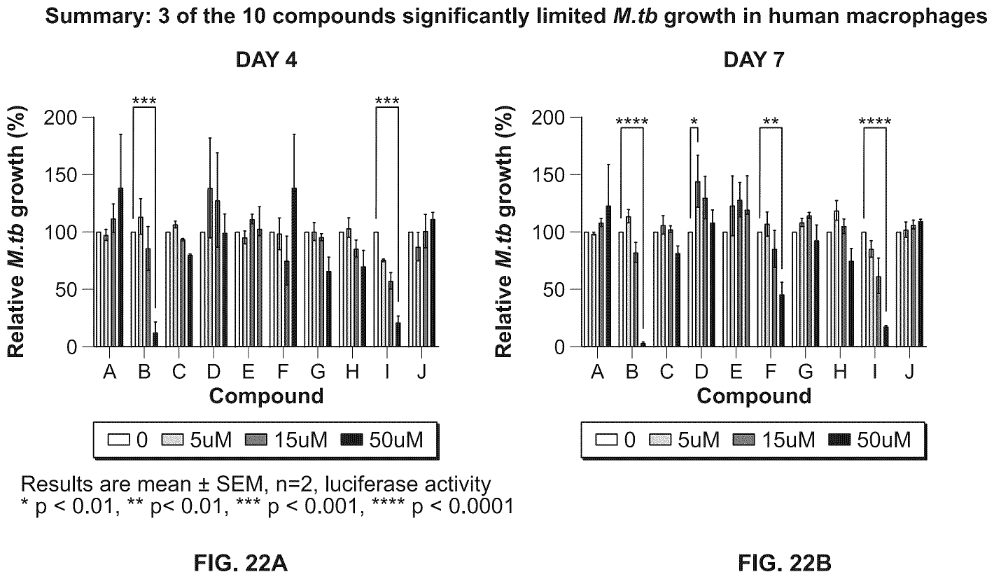

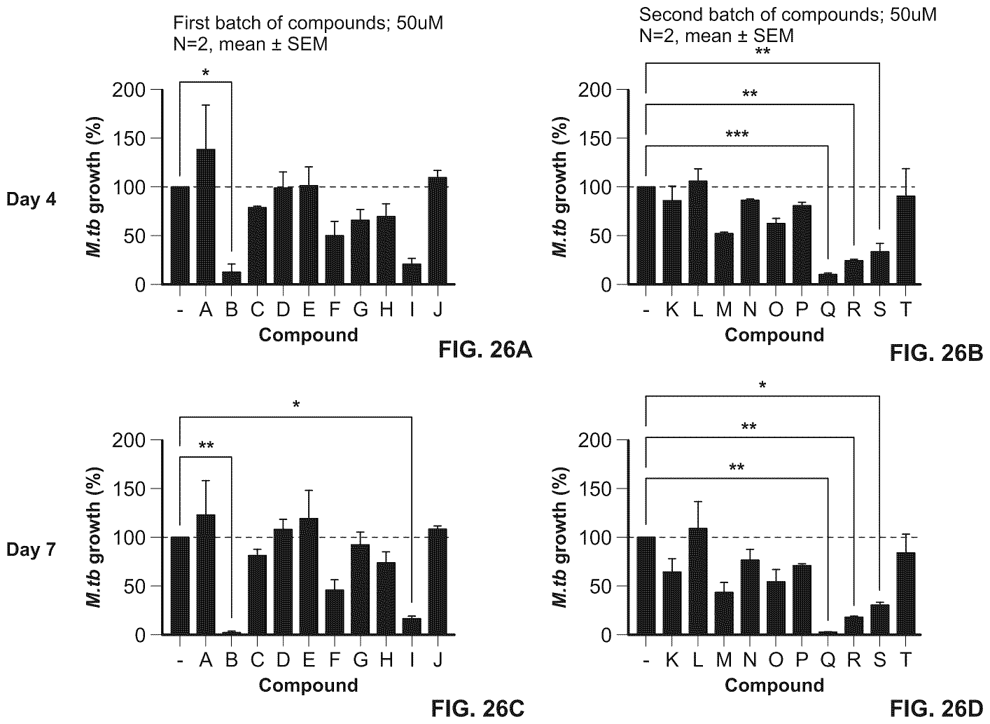

FIGS. 22A-22B are graphical representations of the effect of the 10 compounds on M.tb growth in human macrophages at day 4 and day 7 at different concentrations (0 μM, 5 μM, 15 μM, and 50 μM). FIG. 26A is a graphical representation that summarizes the effect of the 10 compounds (50 μM) on M.tb growth in human macrophages at day 4. For ease, compounds were lettered A-J, Life Chemicals product number is provided in parentheses: A (F5964-0242), B (F6279-0659), C (F5964-0253), D (F1065-0517), E (F0020-1803), F (F5857-5371), G (F5857-5401), H (F0915-2916), I (F0537-0485) and J (F0307-0288). Compounds with predicted binding to NR4A1, 2, or both exhibited varied responses: from control to modestly increasing growth (FIG. 11A). M.tb growth relative to the solvent control (−) are mean±SEM, n=2, **p<0.01, ***p<0.001 relative to (−). The compounds are listed based on which NR4A they are predicted to bind (1, 1 and 2, or 2), compounds with amide or urea groups are in white, and those with carboxylic acids are in gray. The three most potent compounds (B, F, I; confirmed to bind NR4A1/2, see above) contained polar amides or urea groups in addition to large hydrophobic groups. More polar compounds, specifically those with carboxylic acids, were less potent (compounds D, E, H), making them less desirable in future studies. The ability of compounds B and I (the 2 most potent in MDMs) to limit M.tb growth in murine macrophages was evaluated. Excitingly, both compounds significantly reduced M.tb growth in murine macrophages (FIG. 11B).

Additional docking studies were performed by overlaying commercially available compound libraries with B and I, and ten additional compounds were identified with similar structural and electronic properties for further evaluation. Testing the second batch of 10 compounds for anti-M.tb activity and macrophage toxicity in human macrophages. M.tb growth was assessed by M.tb luciferase activity (RLUs) daily. MDM toxicity was assessed by LDH release assay. For ease, compounds were lettered K-T, Life Chemicals product number is also indicated. Eight of the ten compounds significantly limited M.tb growth without causing MDM toxicity. In fact, many reduced M.tb-induced toxicity.

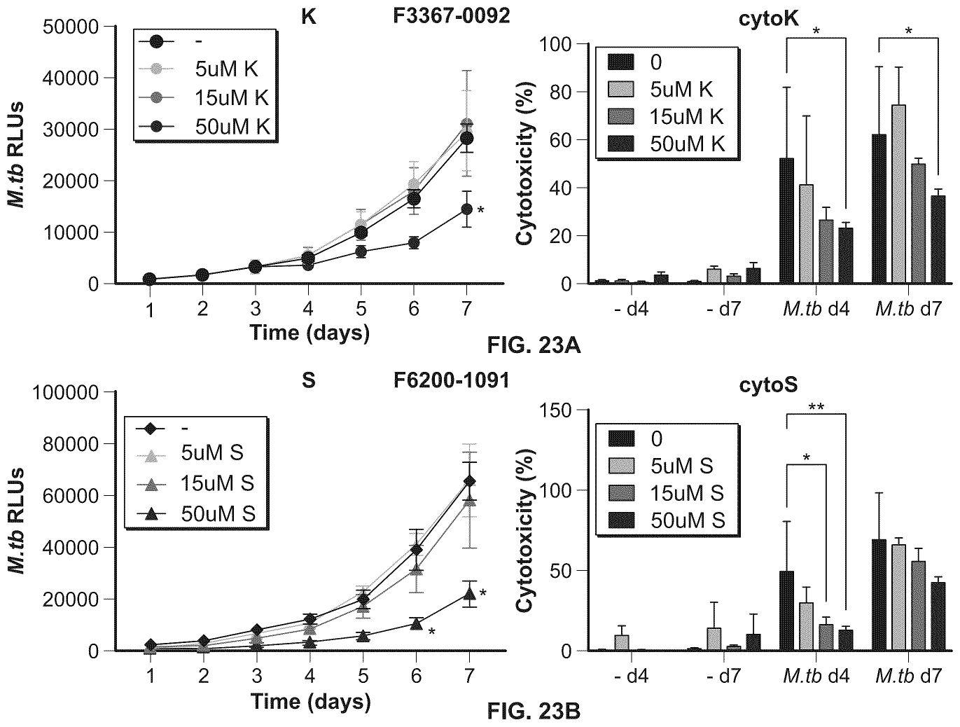

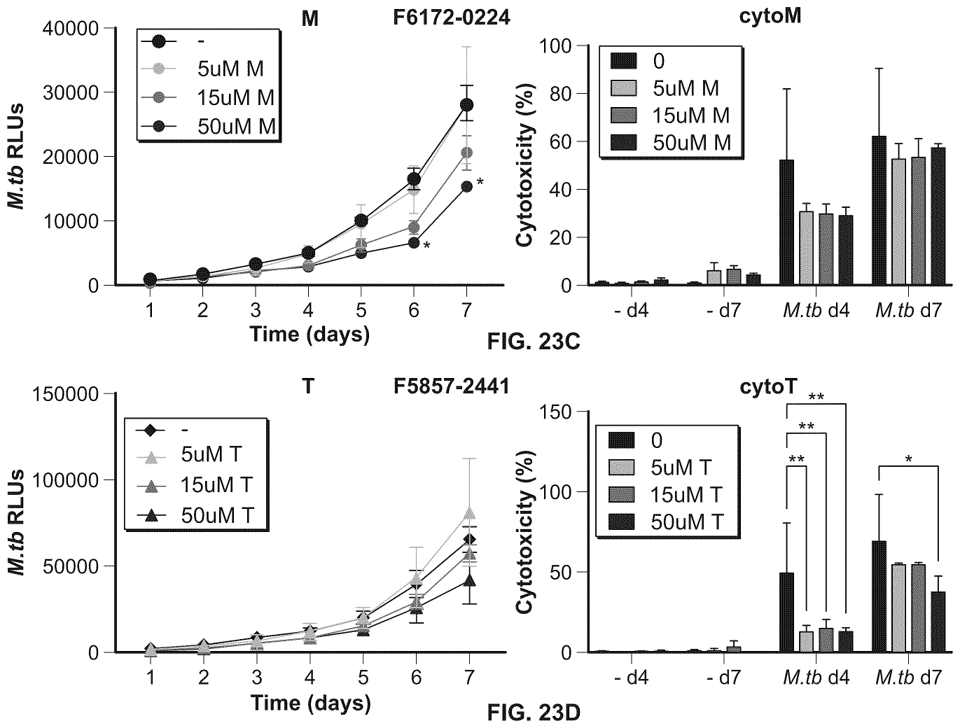

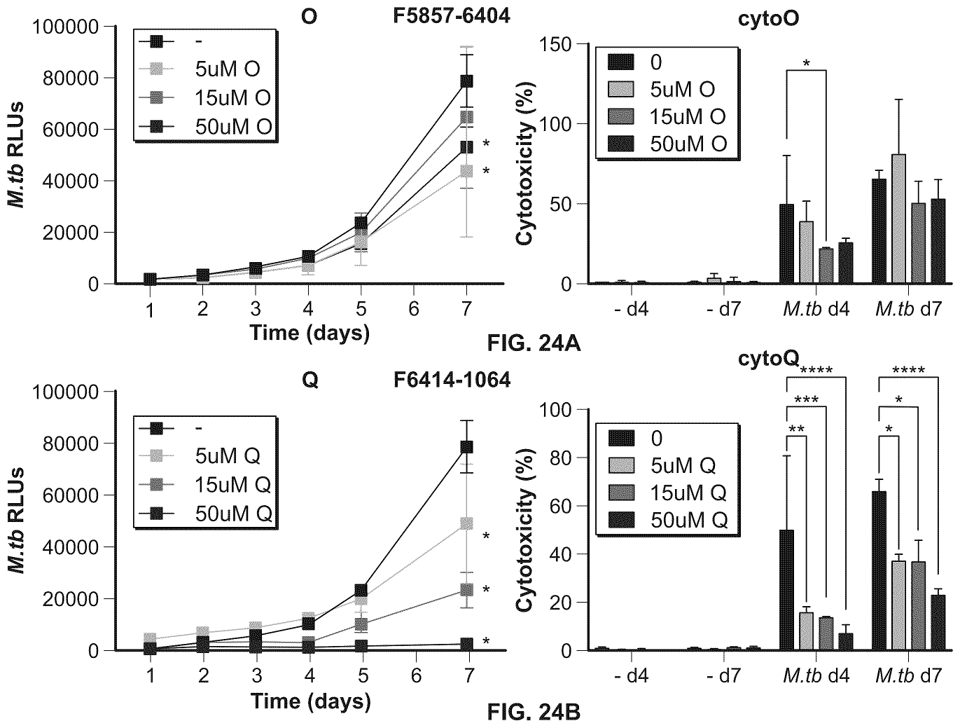

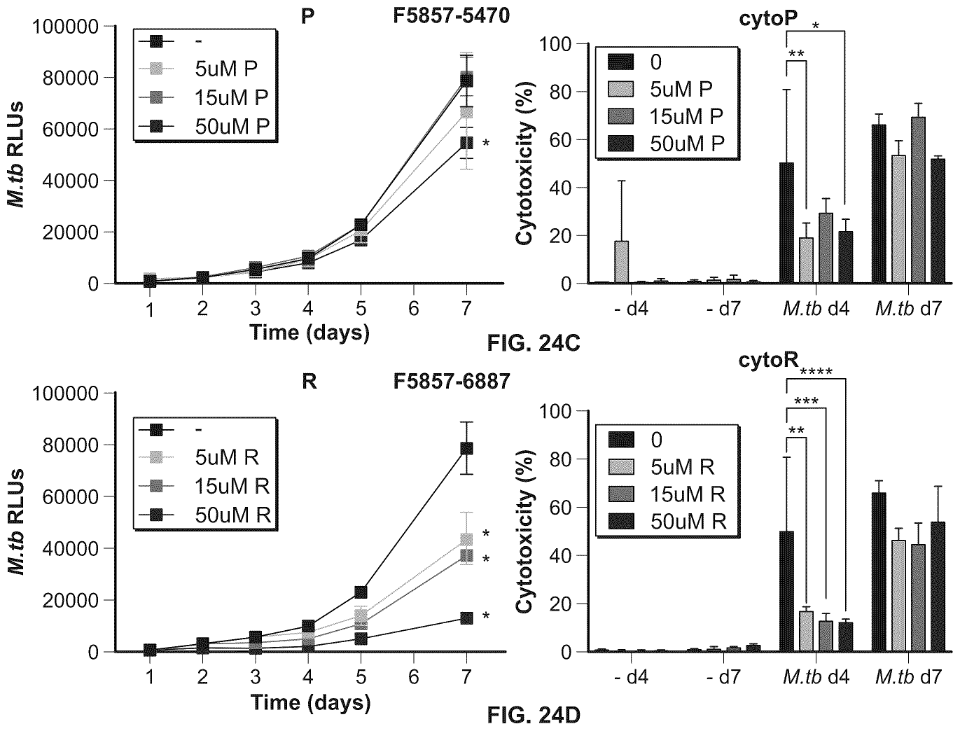

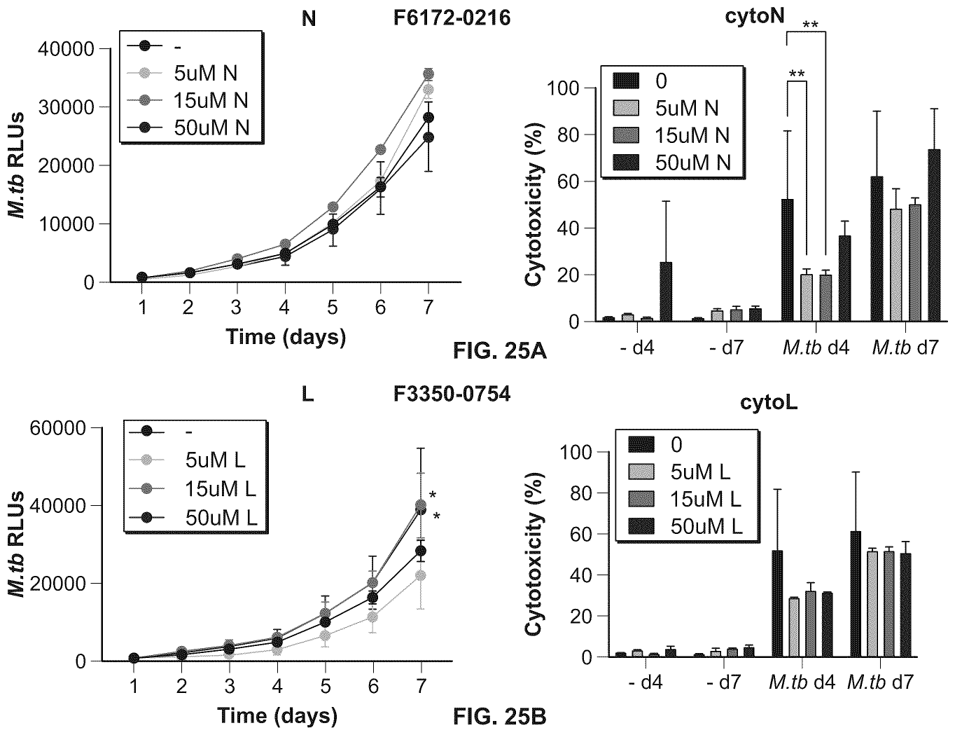

FIGS. 23A-23D, FIGS. 24A-24D, and FIGS. 25A-25B are graphical representations of the second batch of 10 compounds for anti-M.tb activity (left panel) and macrophage toxicity (right panel) in human macrophages. FIGS. 26A-26D are graphical representations of the effect of the 20 compounds on M.tb growth in human macrophages at day 4 (FIGS. 26A-26B) and day 7 (FIGS. 26C-26D) at 50 μM.

As NR4As are host molecules, NR4A agonists are expected to enable macrophages to limit growth of both drug-susceptible and-resistant M.tb. They can likely be used as adjunctive therapy in conjunction with antibiotics, to better control drug-susceptible and-resistant M.tb growth. Some aspects include administering to the subject a therapeutically effective amount of an anti-tuberculosis drug, along with a NR4A agonist. Examples of anti-tuberculosis drugs include first-line anti-tuberculosis drugs, such as isoniazid, rifampin, rifabutin, rifapentine, ethambutol, and pyrazinamide. Other examples of anti-tuberculosis drugs include second-line anti-tuberculosis drugs, such as cycloserine, capreomycin, kanamycin, amikacin, fluoroquinolones (e.g. levofloxacin and moxifloxacin), ethionamide, protionamide, terizidone, and para-aminosalicylic acid.

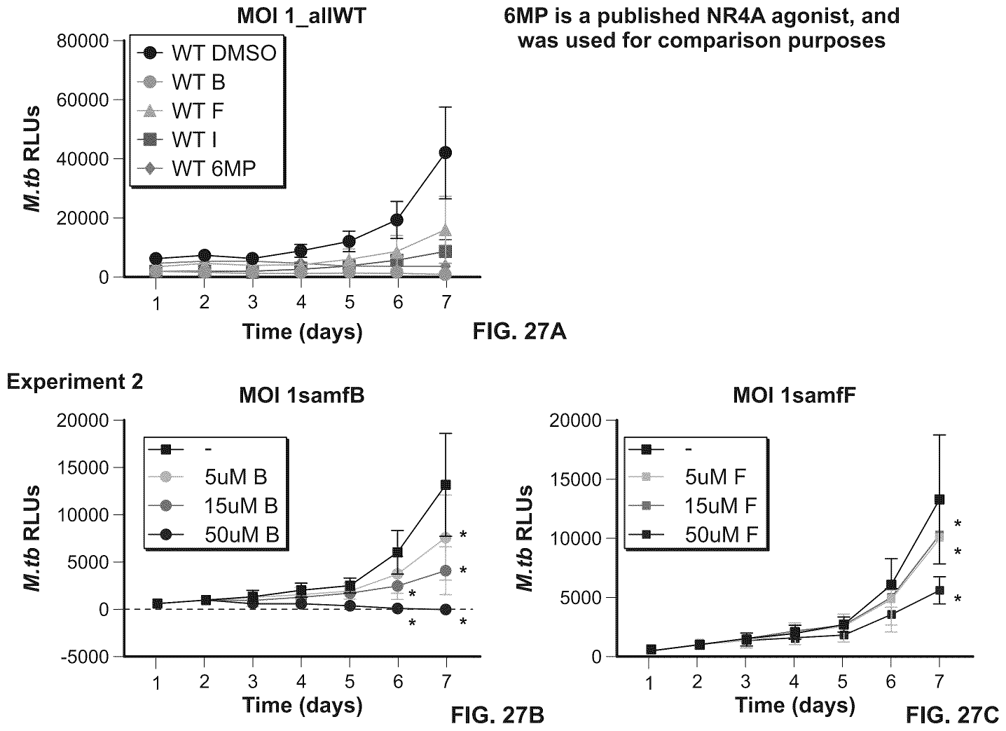

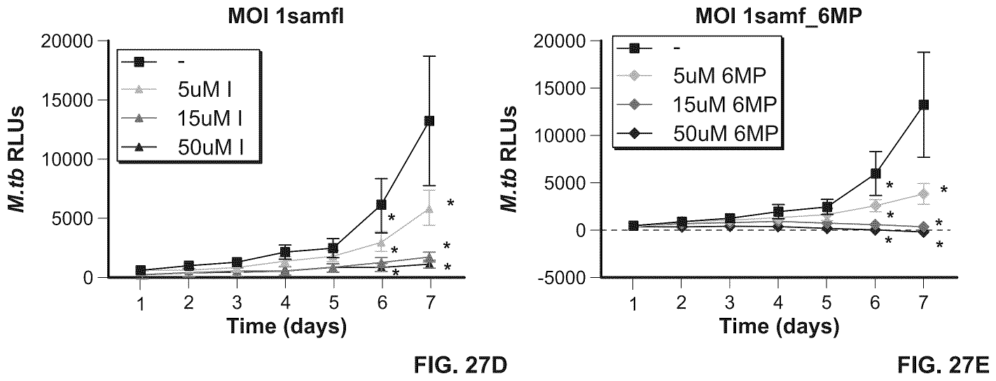

The three most potent compounds from the first batch (B, F, and I) were tested for anti-M.tb activity in murine macrophages. M.tb growth was assessed by luciferase activity daily. FIGS. 27A-27E are graphical representations of the effect of the B, F, and I compounds on anti-M.tb activity in murine macrophages, as assessed by luciferase activity, with 6MP (a NR4A agonist) as a control. Compounds B, F, I are more potent at reducing M.tb in murine macrophages as compared to human macrophages. The murine model is an appropriate model to study impact of NR4A modulators on M.tb infection in vivo.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A method for treating a patient with an infectious or an inflammation-related disease, comprising:
administering a therapeutically effective amount of 3-(adamantan-1-yl)-1-(2-hydroxy-2-phenylpropyl)urea (available at Life Chemicals as F6279-0659) or a pharmaceutically acceptable salt or derivative thereof.

2. A method for treating a patient with an infectious or an inflammation-related disease, comprising:
administering a therapeutically effective amount of N-(3-acetylphenyl)-5,6,7,8-tetrahydronaphthalene-2-carboxamide (available at Life Chemicals as F0537-0485) or a pharmaceutically acceptable salt or derivative thereof.

3. A method for treating a patient with an infectious or an inflammation-related disease, comprising:
administering a therapeutically effective amount of one of N-[(1-hydroxy-2,3-dihydro-1H-inden-1-yl)methyl]naphthalene-1-carboxamide (available at Life Chemicals as F5857-5371), 3,4-difluoro-N-[(2-hydroxy-1,2,3,4-tetrahydronaphthalen-2-yl)methyl]benzamide (available at Life Chemicals as F6414-1064), N-(3-hydroxy-3-phenylpropyl)naphthalene-1-carboxamide (available at Life Chemicals as F5857-6887), N-(2-hydroxy-3-phenylpropyl)naphthalene-1-carboxamide (available at Life Chemicals as F6200-1091) or a pharmaceutically acceptable salt or derivative thereof.

4. The method of claim 1, wherein the infectious disease is caused by Mycobacterium tuberculosis.

5. The method of claim 4, wherein M.tb growth is inhibited by greater than eight percent as compared to a control.

6. The method of claim 4, wherein the method further comprises administering to the subject a therapeutically effective amount of an anti-tuberculosis drug.

7. The method of claim 1, wherein the infectious disease is caused by one or more of Salmonella, Leishmania, Listeria monocytogenes, Francisella tularensis, Yersinia pestis, Histoplasma, Cocciodomycosis, Blastomycocis, or Coxiella burnetii.

8. The method of claim 1, wherein the infectious disease is caused by a virus.

9. The method of claim 1, wherein the inflammation-related disease is one or more of diabetes, Alzheimer's disease, and cancer.

10. The method of claim 2, wherein the infectious disease is caused by Mycobacterium tuberculosis.

11. The method of claim 10, wherein the method further comprises administering to the subject a therapeutically effective amount of an anti-tuberculosis drug.

12. The method of claim 2, wherein the infectious disease is caused by one or more of Salmonella, Leishmania, Listeria monocytogenes, Francisella tularensis, Yersinia pestis, Histoplasma, Cocciodomycosis, Blastomycocis, or Coxiella burnetii.

13. The method of claim 2, wherein the infectious disease is caused by a virus.

14. The method of claim 2, wherein the inflammation-related disease is one or more of diabetes, Alzheimer's disease, and cancer.

15. A method for treating a patient with an infectious or an inflammation-related disease, comprising:
administering a therapeutically effective amount of one or more of F5964-0242, F5964-0253, F1065-0517, F0020-1803, F5857-5401, F0915-2916, F0307-0288, F3350-0754, F6172-0216, F3367-0092, F6172-0224, F5857-2441, F5857-6404, and F5857-5470 (available at Life Chemicals) or a pharmaceutically acceptable salt thereof.

16. The method of claim 15, wherein the infectious disease is caused by Mycobacterium tuberculosis.

17. The method of claim 16, wherein the method further comprises administering to the subject a therapeutically effective amount of an anti-tuberculosis drug.

18. The method of claim 15, wherein the infectious disease is caused by one or more of Salmonella, Leishmania, Listeria monocytogenes, Francisella tularensis, Yersinia pestis, Histoplasma, Cocciodomycosis, Blastomycocis, or Coxiella burnetii.

19. The method of claim 15, wherein the infectious disease is caused by a virus.

20. The method of claim 15, wherein the inflammation-related disease is one or more of diabetes, Alzheimer's disease, and cancer.
